(19) United States
(12) Patent Application Publication
(10) Pub. No.: US 2007/0038226 A1
(43) Pub. Date: Feb. 15, 2007

(54) Embolectomy procedures with a device comprising a polymer and devices with polymer matrices and supports

(76) Inventors: Jason A. Galdonik, Hanover, MN (US); Matthew F. Ogle, Fitchberg, WI (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/494,783

(22) Filed: Jul. 27, 2006

Related U.S. Application Data
(60) Provisional application No. 60/704,099, filed on Jul. 29, 2005.

(51) Int. Cl. A61B 17/26

(52) U.S. Cl. 606/114

(57) ABSTRACT

Embolectomy procedures can be performed under conditions to effectively remove thrombus/emboli with little risk of injuring the vessel wall and while effectively capturing and removing emboli. Desired embolectomy devices present a polymer matrix against the vessel wall. Suction can be used to facilitate the embolectomy process and to remove emboli loosened in the procedure. Some devices combine features to present a polymer matrix against the vessel wall while corresponding providing sufficient support to effectively loosen thrombus. Combinations of embolectomy and/or filter elements can be used.

### CROSS REFERENCE TO RELATED APPLICATIONS

This application claims priority to copending U.S. Provisional Patent Application Ser. No. 60/704,099 to Galdonik et al. filed on Jul. 29, 2005, entitled “Embolectomy Procedures Based on Three Dimensional Filtration Matrix,” incorporated herein by reference.

### FIELD OF THE INVENTION

The invention relates to embolectomy/thrombectomy procedures performed by translation of a embolectomy device to mechanically disrupt emboli/thrombus with the embolectomy device for subsequent removal from a patient's vessel. The invention further relates to systems for performing an embolectomy using improved embolectomy device designs. In some embodiments, improved devices are described that can be used effectively as embolic protection devices in a range of medical procedures in vessels.

### BACKGROUND OF THE INVENTION

An embolus can be any particle comprising a foreign and/or native material, which enters the vascular system or other vessel of the body with potential to cause occlusion of flow, such as blood flow. Emboli can be formed from aggregates of fibrin, blood cells or fragments thereof, collagen, cholesterol, plaque, fat, calcified plaque, bubbles, arterial tissue, and/or other miscellaneous fragments or combinations thereof. Emboli can lodge in the narrowing regions of medium size blood vessels that feed the major organs. Loss of blood flow to surrounding tissue causes localized cell death or micro-infarcts. Cerebral micro-infarcts can cause stroke leading to confusion, disturbance of speech, paralysis, visual disturbances, balance disturbances and even death. In the heart, emboli can cause myocardial infarcts, i.e. heart attacks. Myocardial infarction refers to the death of a section of myocardium or middle layer of the heart muscle. Myocardial infarction can result from at least partial blockage of the coronary artery or its branches. Blockage of capillaries associated with the coronary arteries can result in corresponding micro-infarctions/micro-infarcts. Resulting impairments are frequently short term but can be permanent.

In some contexts, thrombus has been used to refer specifically to clots generally comprising fibrin. However, as used herein with respect to removal from a vessel, thrombus is used broadly to refer to any debris within a vessel that is restricting flow. Thus, thrombus is used interchangeably with emboli. Thrombus can result in undesirable restriction of flow within the vessel. In addition, release of thrombus from a particular location can result in a more serious blockage of flow downstream from the initial release location. Foreign material in the stream of flow can cause turbulence or low flow. Such flow conditions have been shown to increase rates of infection.

Disease states including, for example, arteriosclerosis and deep vein thrombosis, aging and even pregnancy can cause build up of plaque and fibrin on vessel walls. Anything that loosens or breaks up this plaque can generate emboli/thrombus. The clinical ramifications of emboli are staggering. Emboli generated from arteriosclerosis of the carotid artery alone cause 25% of the 500,000 strokes that occur yearly in the United States (2002 American Heart Association And Stroke annual statistics).

Ironically, the surgical interventions used to remove or bypass the plaque of arteriosclerosis (e.g., balloon dilatation angioplasty, endarterectomy, bypass grafting and stenting) can themselves disrupt plaque. One of the most common cardiovascular interventions is coronary artery bypass grafting (CABG). Historically, 10-20% of all CABG interventions generate emboli large enough to cause myocardial infarcts. This is particularly true when the graft used is of saphenous vein origin. But CABG is not the only procedure with potential to generate emboli. In fact, doppler ultrasound shows evidence of micro-embolization in almost all cardiac intervention cases. Of the over 1.8 million intervention procedures performed annually, greater than 10% result in neurocognitive disturbance and/or ischemic event. These impairments are frequently short term, but can be permanent.

Surgical procedures for the treatment of renal artery stenosis can also generate emboli. There is clinical evidence to suggest that 36% of those treated suffer arterioloar nephrosclerosis caused by atheroemboli. Five-year survival of patients with atheroembolic events is significantly worse than of patients without atheroemboli (54% vs. 85% respectively) [Krishmamurthi et al. J Urol. 1999, 161:1093-6].

### SUMMARY OF THE INVENTION

In a first aspect, the invention pertains to a method for the removal of thrombus with an embolectomy device comprising a guide structure and a plurality of fibers operably connected to the guide structure. The method comprises moving the embolectomy device within a vessel of a patient to dislodge thrombus with the embolectomy device. Suction is applied within the vessel to remove at least a portion of the dislodged thrombus.

In a further aspect, the invention pertains to a method for the removal of thrombus with an embolectomy device comprising a guide structure, an embolectomy device comprising a polymer matrix and a filter comprising a three-dimensional filtration matrix having a deployed configuration with the filtration matrix extending across the lumen of a vessel. The method comprises moving the embolectomy device within a vessel of a patient to dislodge thrombus while the filter is in its deployed configuration at a fixed location within the vessel.

In another aspect, the invention pertains to a biocompatible filtration device comprising a guide structure having a proximal end and a distal end, a three dimensional filtration matrix connected near the distal end of the guide structure and a porous filtration membrane connected to the guide structure distal to the three dimensional filtration matrix.

In additional aspects, the invention pertains to a biocompatible filtration device comprising a guide structure having a proximal end and a distal end, a three dimensional filtration matrix connected near the distal end of the guide structure and a support structure interfaced with the three dimensional filtration matrix to form a filter element having a deployed configuration and a recovery configuration. In the deployed configuration, the three dimensional filtration matrix extends outward from the support structure with respect to the filtering function of the device relative to the axis of the guide structure.

Moreover, the invention pertains to a biocompatible embolectomy device comprising a guide structure having a proximal end and a distal end, a plurality of polymeric fibers connected near the distal end of the guide structure and a support structure. Generally, the fibers interface with the support structure to reinforce the fibers, and the fibers have a deployed configuration and a recovery configuration. In the deployed configuration, the polymeric fibers extend outward from the support structure relative to the axis of the guide structure.

In addition, the invention pertains to an embolectomy device comprising a tubular catheter having a distal end and a proximal end with a lumen extending from the proximal end to an aspiration port at or near the distal end. An aspiration apparatus can be operably connected at or near the proximal end of the tubular catheter. A plurality of fibers connected to the surface of the catheter at a localized section within 10 centimeters from the distal end of the catheter and extending outward when unconstrained.

Furthermore, the invention pertains to a method for removing debris from a vessel of a patient in which the method comprises sweeping an embolectomy device through a deployed stent within a patient's vessel and applying suction to remove debris loosened by the embolectomy device.

### BRIEF DESCRIPTION OF THE DRAWINGS

FIG. 1 is a schematic depiction of a system for performing the embolectomy procedure described herein.

FIG. 2 is a schematic side view of an embolectomy system having an embolectomy device and a separate filter.

FIG. 3A is a side view of a device comprising a polymer matrix, which can be a three-dimensional filtration matrix, and a basket-type filter element, in which the polymer matrix and the basket filter element are in a spaced apart configuration.

FIG. 3B is a side view of a device comprising a polymer matrix, which can be a three-dimensional filtration matrix, and a basket-type filter element, in which the polymer matrix is positioned at the opening into the basket filter element.

FIG. 3C is a side view of a device comprising a polymer matrix, which can be a three-dimensional filtration matrix, and a basket-type filter element, in which the polymer matrix is at least partially within the interior of the basket filter element.

FIG. 4 is a sectional side view of a particular embodiment of an integrated guide structure and an embolectomy device.

FIG. 5 is a side view of the integrated device of FIG. 4.

FIG. 6 is a side view of the corewire of the integrated device of FIG. 4.

FIG. 7 is a side view of the device of FIG. 4 following expansion of the embolectomy device.

FIG. 8 is a side view of an interventionary medical device comprising a polymer matrix interfaced with a support structure connected to a guide structure.

FIG. 9 is a side view of an interventionary medical device comprising fibers interfaced with a support structure connected to a guide structure.

FIG. 10A is a side view of a rapid exchange embolectomy catheter with fiber elements associated with the exterior of the catheter body such that the single device can be used to free emboli and use suction to remove the emboli from a vessel, in which the suction ports are placed proximal to the fibers, with hidden structure shown in phantom lines.

FIG. 10B is a side view of a rapid exchange embolectomy catheter with fiber elements associated with the exterior of the catheter body such that the single device can be used to free emboli and use suction to remove the emboli from a vessel, in which the suction port is at the distal end of the catheter, with hidden structure shown in phantom lines.

FIG. 10C is a side view of a rapid exchange ebmolectomy catheter with fiber elements associated with the exterior of the catheter body and suction ports at the base of the embolectomy device in a distal position with a design specific for removing a clot that is difficult to pass in the vessel, with hidden structure shown in phantom lines.

FIG. 10D is a side view of a rapid exchange embolectomy catheter with a break in the catheter structure with a fiber based embolectomy structure connecting the two sections of the catheter in which the embolectomy device is in a delivery configuration with the fibers outstretched, with hidden structure shown in phantom lines.

FIG. 10E is a side view of the rapid exchange embolectomy catheter of FIG. 10D in which the embolectomy structure is in a deployed configuration with the fibers flared outward from the axis of the catheter, with hidden structure shown in phantom lines.

FIG. 11 is a side sectional view of a vessel with an embolectomy device having a polymer matrix for performing an embolectomy procedure.

FIG. 12 is a side view of the vessel and embolectomy device of FIG. 11 in which the device has been moved downstream to loosen emboli/thrombus for removal from the vessel.

FIG. 13 is a sectional view of a patient's vessel with an embolectomy device being delivered past a lesion with thrombus/emboli.

FIG. 14 is a sectional view of the embolectomy of FIG. 13 deployed downstream from the thrombus/emboli.

FIG. 15 is a sectional view of the embolectomy device of FIG. 13 converted to a deployed configuration downstream from the thrombus/emboli.

FIG. 16 is a sectional view of the embolectomy device of FIG. 13 following movement of the embolectomy device downstream past the location of the thrombus/emboli in which suction is applied for at least a portion of the time that the device is moved downstream.

FIG. 17 is a sectional view of an aspiration catheter adjacent the embolectomy device of FIG. 13 following movement of the device but prior to recovery of the embolectomy device.

FIG. 18 is a sectional view depicting the transition of the embolectomy device of FIG. 13 transformed to a recovery configuration while suction is applied.

FIG. 19 is a sectional view of the embolectomy device withdrawn into the aspiration catheter of FIG. 17 for removal of the embolectomy device from the patient.

FIG. 20 is a sectional view of an embolectomy system deployed in a vessel with a lesion in which the system comprises an embolectomy device and a separate filter.

FIG. 21 is a sectional view of the embolectomy system of FIG. 20 in which the embolectomy device and the filter are in a deployed configuration.

FIG. 22 is a sectional view of the embolectomy device of FIG. 20 in which the embolectomy device has been moved to perform the embolectomy procedure while the filter remains at its original deployed position.

FIG. 23 is a sectional view of the embolectomy system of FIG. 20 following the performance of the embolectomy procedure in which the embolectomy device and the filter have been converted to respective recovery configurations.

FIG. 24 is a side sectional view of an alternative embodiment depicting a sheath placed to assist with recovery of the embolectomy device following the procedure depicted in FIG. 22.

FIG. 25 is a side sectional view of the embolectomy device retracted into the sheath depicted in FIG. 24.

FIG. 26 is a side sectional view showing an embolectomy device of FIG. 10A positioned to perform an embolectomy procedure within a vascular stent.

FIG. 27 is a side sectional view of an embolectomy device positioned with a suction catheter to perform an embolectomy procedure within a vascular stent.

FIG. 28 is a side sectional view of the devices of FIG. 27 withdrawn within a vascular stent during the performance of an embolectomy procedure.

### DETAILED DESCRIPTION OF THE INVENTION

Improved embolectomy devices present polymer surfaces against the vessel wall that are less likely to cause trauma to the vessel wall relative to devices with rigid structures contacting the wall. Generally, an improved device can present a polymer matrix along the vessel wall that provides a desired level of function with respect to freeing thrombus within the vessel. In some embodiments, the device comprises fibers that have a deployed configuration with portions of the fiber along the vessel wall following deployment. A polymer matrix can be combined with other cooperative structures, such as a filter and/or support elements. Movement of the passive embolectomy device can be used to dislodge debris within the vessel without any powered components being within the patient. The device may entrap at least some of the debris, although a separate associated filter can be used to capture emboli that escapes the embolectomy device. In addition, suction can be used to remove emboli as the device is moved and/or at other times in the procedure, such as for retrieval of the embolectomy device.

Once a region for treatment is identified, the embolectomy device is delivered to the selected location. In some embodiments, the embolectomy device is delivered into the patient using a less invasive, percutaneous procedure, usually catheter-based, through a small incision. In other embodiments, the embolectomy device can be delivered in a surgical procedure that provides entry into the vessel upon exposure of the vessel through the parting of tissue. The embolectomy device can be delivered to an initial location within the patient in a low profile delivery format and expanded to its deployed configuration within the vessel. Generally, relatively unchanged flow can be maintained in the vessel during the procedure. In some embodiments, the embolectomy procedure can be combined with other treatment procedures, such as angioplasty, to diminish or eliminate restrictions to flow within the vessel. The embolectomy procedures and corresponding devices described herein are generally used on mammalian patients, in particular humans. Similarly, these procedures and devices can be used in any blood vessel, urinary vessel or other vessel of the patient.

The treatment system for performing the embolectomy procedure generally comprises an embolectomy device attached to a tether. In some embodiments, the treatment system can further comprise a catheter, such as a suction catheter, and/or a distinct filter element. In embodiments of particular interest, the embolectomy device in a deployed configuration presents a polymer matrix, such as an elastic polymer, at least along its outer surface relative to the axis of the tether. Thus, the deployed embolectomy device can present a less harsh material along the vessel wall during use to avoid damaging the vessel during the procedure. At the same time, the device can be effective to loosen debris for removal from the vessel, and the system overall can effectively entrap and remove thrombus to improve flow through the vessel. Also, a loosely configured network of fibers acts independently to fully oppose the vessel wall, even if the vessel wall is diseased or non-circular.

Embolectomy devices based on selected polymer matrices, such as a bundle of fibers, can provide desired non-harsh surfaces at a vessel wall while being effective to loosen debris, such as thrombus, within a vessel. Suitable polymer matrices may or may not form three-dimensional filtration matrices depending on the density or extent of the matrix. In particular, the embolectomy device can have a polymer matrix with a more porous structure than a filter matrix if other features of the system are provided to capture and/or remove emboli loosened by the embolectomy device. Suitable porous polymer matrix structures include, for example, suitable hydrogels that expand into a porous matrix upon exposure to liquids in a vessel, compressed porous polymer matrices and the like. Alternatively or additionally, the polymer matrix can comprise fibers. The polymer matrix may or may not extend around the circumference of the tether since the procedure may be directed along only a portion of a vessel wall. However, a polymer matrix that extends around the entire circumference can be convenient through the elimination of orientation of the device at deployment.

Fibers can be used to form convenient embolectomy devices. In particular, the fibers can form a deployed embolectomy device that has a suitable polymer along a vessel wall. At the same time, the structure, number and positioning of the fibers and other components of the device can be designed for effective embolectomy procedures. In particular, the fibers can be made at a selected thickness and from a selected material such that the device provides a desired level of performance during the procedure. While in some embodiments, the fibers can be woven, it can be convenient to use a bundle of unwoven fibers that are bent to extend outward in a deployed configuration.

While it is desirable to present a softer surface to the vessel wall, if the porous matrix of the embolism protection device is too flexible, the embolism protection device may not be able to apply sufficient force onto some thrombus to disrupt the substance. To provide additional reinforcement to the embolectomy device, the polymer matrix can be associated with struts, frame or other support structure. In some embodiments, the support structure does not extend outward from the axis of the tether as far as the deployed polymer matrix so that generally the polymer matrix contacts the vessel wall. Nevertheless, a support structure can provide additional mechanical stability to the embolectomy device as it is moved within the vessel so that greater force can be applied with the polymer matrix to thrombus to dislodge the thrombus. For example, a support structure can be formed form a spring metal so that the support structure can be transitioned between a deployed configuration and a delivery/removal configuration. In some embodiments, fibers are interfaced with the support structure to achieve desired structural stability.

In general, the tether can be any suitable device that can be delivered into the vessel while supporting the embolectomy device. The tether may or may not be involved in conversion of the embolectomy device between different configurations. In some embodiments, the tether comprises a guide structure. In other embodiments, the tether comprises an overtube that rides over a guide structure. In further embodiments, the tether comprises a catheter, which can be an aspiration catheter. In some embodiments of particular interest, the tether comprises a first element and an overtube or the like that can move relative to the first element to transition the embolectomy device between a delivery configuration and a deployed configuration.

In some embodiments of interest, the tether comprises an integrated guide structure comprising a corewire and an overtube. In some embodiments, a bundle of fibers have one end fastened to the corewire and their other end to the overtube so that relative motion of the corewire and overtube can straighten the fibers to a delivery/recovery configuration or bend the fibers to a deployed configuration with the central of the fibers extending outward away from the axis of the integrated guiding structure. Similar structures can be formed with a tube and an actuation element, such as an overtube, riding within or over the tube. In these embodiments, the fibers have one end secured to the tube and their other end secured to the actuation element so that relative movement of the tube and actuation element can be used to deploy the fibers. In some embodiments, the tube, for example, can ride with little clearance over a guide wire or an integrated guiding structure. In other embodiments, the tube can be a catheter with an open central lumen, such as a suction catheter. The actuation element may or may not need not form a tubular enclosure since it does not control flow of any liquid, as long as it can ride within or over the tube and transmit relative movement along the length of the tube. Alternative structures for the actuation element are discussed further below.

The embolectomy device can entrap the loosened emboli during the embolectomy procedure. For these embodiments, the polymer matrix of the embolectomy devices can form filtration matrices. However, whether or not the embolectomy device has a filtration matrix, it may be desirable for the embolectomy system to have a downstream filtration device to provide embolic protection against any emboli that flow downstream from the polymer matrix during the procedure.

A wide variety of filter devices can be adapted for placement down stream from the embolectomy device. For example, basket-type filter devices are commercially available. Commercially available filtration devices include, for example, the RX Accunet™ Embolic Protection System, available from Guidant, Indianapolis, Ind. This Guidant filter is formed from a nickel-titanium alloy in a mesh. Also, Boston Scientific (Boston, Mass.) markets FilterWire EZ™ Embolic Protection System. The Boston Scientific device has a polyurethane filter. See also, U.S. Pat. No. 6,695,813 to Boyle et al., entitled “Embolic Protection Devices,” and U.S. Pat. No. 6,391,045 to Kim et al., entitled “Vena Cava Filter,” both of which are incorporated herein by reference.

For appropriate embodiments, a three dimensional filtration matrix, such as a fiber matrix, provides for improved emboli capture due to the ability to trap particles of debris within the matrix and/or on the surface of the matrix without blocking the flow. Specifically, the three-dimensional flow matrix within the filter provides alternative flow paths through the filter matrix while decreasing or eliminating a pressure drop across the filter. The three-dimensional filtration matrix, for example, can be formed from fibers and/or from a porous polymer matrix. In addition, fiber-based embolic protection devices have distinct advantages over basket type designs. For example, fiber-based devices can conform to irregular vessel walls, and they are gentle on the vessel with less likelihood of inducing damage to the wall. In addition, the fiber-based devices can have less lateral extent within the vessel such that changes in direction of the vessel and/or side vessels can be less of an issue.

In some embodiments, the fibers are attached to an integrated guiding structure in which the relative position of a corewire and overtube can be used to deploy and recover the filter device. The embolectomy device can be delivered over the integrated guiding structure. The fibers in the filter can be surface capillary fibers. The number and length of the fibers can be selected based on the range of vessel sizes that the device is intended for deployment. Fiber-based embolism protection devices are described further in copending U.S. patent application Ser. Nos. 10/795,131 to Galdonik et al., entitled “Fiber Based Embolic Protection Devices,” and 11/072,001 to Galdonik et al., entitled “Steerable Device Having a Corewire Within a Tube And Combination With a Functional Medical Device,” both of which are incorporated herein by reference.

In some embodiments, a fiber-based device is placed proximal to a basket-type filter. The fiber elements can spaced apart from the basket, at the mouth of the basket and/or within the basket volume. In some of these configurations, the basket may not extend quite to the vessel wall since a fiber filter device has dense fibers near the vessel wall. Thus, the fibers present a less traumatic surface to the vessel wall while some of the structural features of the basket can be used to provide greater structural stability to the composite filter, which can be especially useful in larger vessels. These combined devices can be used for embolectomy procedures, or they can be used embolic protection devices during other procedures. In some embodiments, the advantages of both the fiber based devices and the basket devices can be combined in the composite devices.

A catheter can be used to facilitate the embolectomy procedure. The catheter can be selected to have an appropriate size to bring the catheter a short distance proximal to the treatment location. Following the procedure, the embolectomy device and/or a filter can be drawn into the catheter for removal from the patient. Catheters designed for removal of an embolic protection device are described further in copending U.S. patent application Ser. No. 10/854,920 to Galdonik et al., entitled “Emboli Filter Export System,” incorporated herein by reference. The catheter can comprise a curved tip or delivery structures to facilitate delivery of the device with less chance of catching of the catheter during delivery. Improved catheter designs are described further in copending U.S. patent application Ser. No. 11/207,169 to Baldenow et al., entitled “Tracking Aspiration Catheter,” incorporated herein by reference. In some embodiments, the catheter can provide suction to facilitate removal from the vessel of emboli generated during the procedure. Appropriate suction devices, such as syringes, can be used to supply desired levels of suction.

The embolectomy devices described herein can provide a basis for emboli/thrombus removal by moving the device in the vicinity of debris. Movement of the device in an upstream and/or downstream direction can dislodge thrombus. If the embolectomy device is moved in an upstream direction, the natural flow in the vessel facilitates collection of the dislodged emboli in the embolectomy device if the device has a suitable design. However, in some embodiments, a separate filter is placed downstream form the embolectomy procedure to capture any released emboli. The embolectomy devices described herein are passive in the sense that the embolectomy components within the patient are not powered. Thus, the embolectomy devices do not have motorized abraders and do not deliver energy, such as electrical or acoustic forces within the vessel. The forces to perform the embolectomy procedure are all supplied by movement of the tether from outside the patient to move the embolectomy device within the vessel.

Generally, the embolectomy device is delivered and deployed within the vessel in the vicinity of identified thrombus. Suction can be applied while the embolectomy device is being moved within the vessel to collect emboli dislodged through movement of the device. Suction can be started before moving the device and/or continued after stopping moving the device. Similarly, suction can be applied for only a portion of the time that the embolectomy device is moved.

Additionally or alternatively, suction can be applied during the recovery of the embolectomy device and/or a separate filter with entrapped emboli. Also, suction can be applied while the embolectomy device is transitioned from a deployed configuration to a recovery configuration and/or while the embolectomy device is being drawn into the catheter tip. Similarly, if a separate filter or other embolic protection device is used, suction can be used during the recovery process of this device.

Embolectomy System

The system for performing the embolectomy procedure comprises an embolectomy device attached to a tether. The embolectomy device generally comprises a polymer matrix. The tether attached to the embolectomy device can be, for example, a guide structure, such as a guide wire or an integrated guiding device. Optionally, two or more tethers can be used. The embolectomy device may or may not comprise a filtration matrix. Whether or not the embolectomy device has a filtration matrix, the system can further comprise a filter, i.e., an embolic protection device, separate from the embolectomy device. In some embodiments, a suction catheter can be used to capture at least some of the emboli involved in the procedure. If desired, other treatment structures can be used for performance of additional procedures within the vessel in conjunction with the embolectomy device. Some of the embolectomy device designs can be used effectively also as embolic protection devices, which can be used without an embolectomy device.

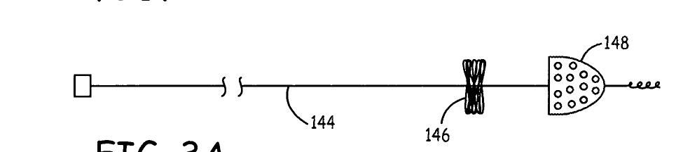

Referring to FIG. 1, a system for performing the embolectomy procedure is shown schematically. Embolectomy system 100 comprises embolectomy structure 102, optional separate filter element 104, optional aspiration catheter 106 and optional additional treatment structure 108. Embolectomy structure 102 comprises an embolectomy device 110, tether 112 and an optional back-end tool 114. Back end tool 114 can be used to actuate the embolism protection device and/or to facilitate movement of the embolectomy device within the vessel. Separate filter element 104 can be associated with the embolectomy structure 102 so that they can be used as a unitary structure or separately deployed and manipulated. Aspiration catheter 106 generally comprises a tubular shaft 116 with an aspiration lumen and a suction device 118, such as a syringe, pump or the like. Additional treatment structure 108 can be, for example, an angioplasty balloon, a stent deployment tool, a second embolectomy device or any other suitable treatment device that can be delivered into a patient's vessel. In some embodiments, aspiration catheter 106 and/or additional treatment structure 108 can be delivered over the tether.

In some embodiments, filter element 104 can be attached to a guide structure that forms part of tether 112 or integrates with tether 112. Filter element 104 can comprise a three dimensional filter structure. In additional or alternative embodiments, filter element 104 can be a basket type filter element with a perforated sheet of filter material. Filter elements that combine a three dimensional filtration matrix along with a basket-type filtration membrane can be effectively used as embolectomy devices, as a filter to be used along with an embolectomy device or as a filter/embolic protection device to be used in other contexts. Referring to FIG. 2, embolectomy structure 130 comprises a back-end tool 132, a guide structure 134, an embolectomy device 136 and a filter element 138. The guide structure can be designed so that embolectomy device 136 and filter element 138 are fixed at a set distance apart. If embolectomy device 136 and filter element 138 are fixed a set distance apart, filter element 138 moves along with embolectomy device 136 as the embolectomy device is moved to perform the embolectomy procedure. In these embodiments, an actuation structure incorporated into guide structure 134 can actuate simultaneously embolectomy device 136 and filter element 138.

In alternative embodiments, guide structure 134 can be designed to provide for the optional relative movement of embolectomy device 136 and filter element 138. In these embodiments, filter element can be left in one position while the embolectomy device is moved to perform the embolectomy procedure, as described further below with respect to the performance of the procedures. In these embodiments, guide structure 134 can comprise on an embolectomy overtube that is connected to the embolectomy device 136, and this embolectomy overtube rides over an inner guide structure that is operably connected with filter element 138. The inner guide structure and/or the embolectomy overtube can comprise appropriate structure to actuate respectively the embolectomy device and/or the filter from a collapsed configuration to a deployed configuration.

As noted above, a separate filter element can be a basket type filter structure. In some embodiments, it is desirable to combine a basket filter element with a device that has a three dimensional filtration matrix, which is referred to for convenience as a matrix filtration device. This structure can be advantagously used as an embolic protection device to be used in general circumstances where emboli are expected to be generated and not just for embolectomy procedures, although the device can be effectively used for embolectomy procedures.

Different relative positions of a matrix filtration device and a basket filter element are shown in FIGS. 3A-3C. Referring to FIG. 3A, guide structure 144 is attached to matrix filtration device 146 and basket filter element 148 in a spaced apart configuration. In some embodiments, the relative position of matrix filtration device 146 and basket filter element 148 can be varied during the procedure, while in other embodiments the relative position is fixed. In contrast, in FIG. 3B guide structure 154 is attached to matrix filtration device 156 and basket filter element 158, in which the matrix filtration device is positioned at the opening of the basket filtration element. Furthermore, in FIG. 3C guide structure 164 is attached to a matrix filtration device 166 and a basket filtration device 168 in which matrix filtration device 166 is within the internal volume of basket filtration device 168. The interior of a basket filtration device can be roughly determined by placing a cap over lip of the filter. In addition, there can be intermediate embodiments between the embodiments of FIG. 3B and FIG. 3C in which the matrix filtration device is partly at the opening and partly in the interior of the basket filtration device. For the embodiments in FIGS. 3B and 3C, the basket filtration device is not separate from the matrix filtration device and relative position of matrix filtration devices 156, 166 are generally fixed relative, respectively, to basket filtration devices 158, 168.

A basket-type filter generally has a filtration membrane having pores drilled, woven, molded or otherwise formed through the two-dimensional membrane. The size of the pores can be selected to allow passing beneficial blood components while blocking emboli with sizes exceeding the pore sizes. The shape of the basket can have the shape of a wind sock or similar shape. The expanded surface area of the basket reduced clogging of the membrane for a particular loading of emboli. Suitable sizes of the pores can be determined for a particular application. For general applications in blood vessels, pores with a diameter from 50 microns to 250 micron can be suitable.

As noted above, the embolectomy device can comprise a polymer matrix, which may or may not form a filtration matrix. The polymer matrix provides a polymer surface for contacting the vessel with a low chance for causing injury to the vessel. In general, if there are other accommodations for capturing and removing the emboli, such as a separate filter and/or a suction catheter, the embolectomy device does not need to function well or at all as a filter. Generally, the embolectomy device is used to loosen emboli for removal from the vessel. The polymer matrix can comprise a porous polymer material, fibers or other polymer segments.

In some embodiments, the embolectomy device comprises a three-dimensional filtration matrix, and in additional or alternative embodiments, a separate filter device is used with a three dimensional filtration matrix. A three-dimensional filtration matrix comprises a network of interconnected and circuitous flow pathways through a three-dimensional mass of material. The network of flow passageways through a three-dimensional filtration matrix may or may not be random. The interconnected and circuitous flow pathways are generally significantly branched. Thus, a three-dimensional filtration matrix is distinctly different from a perforated structure in which holes drilled or otherwise formed in a sheet of material are disconnected, generally unbranched, passages through the material that functions as a two dimensional filtration membrane. In general, a polymer matrix becomes a three-dimensional filtration matrix if the density and porosity are appropriate for performing the filtration function.

In some embodiments, polymer matrices have an expanding structure that incorporate material, such as hydrogels and/or shape memory fibers. For example, a device can comprise a polymeric substrate (media, sponge), especially an expandable polymer, such as a swelling polymer, a memory polymer or a compressed polymer. While shape memory of a polymer may just favor an expanded configuration, the shape memory can induce a desirable expanded shape. Suitable shapes can be, for example, a generally conical shape or other curved shape that tend to collect debris at the center of the device.

Specifically, in some embodiments, the polymer matrices described herein can comprise a swelling polymer that expands, generally spontaneously, upon contact with an aqueous solution, such as blood or other body fluids. Swelling is considered broadly in terms of significant changes in dimension due to absorption or other intake of fluid/liquid into the structure of the material, such as with a sponge, a hydrogel or the like. Hydrogels are generally hydrophylic polymers that are nevertheless not soluble in aqueous solutions. Generally, hydrogels are crosslinked to prevent them from being soluble. Embolism protection devices comprising a swelling polymer, such as hydrogels and/or fibers, for example, shape memory fibers, are described further in copending U.S. patent application Ser. No. 10/414,909 to Ogle, entitled “Embolism Protection Devices,” incorporated herein by reference. This application also describes the delivery of a bioactive agent in conjunction with an embolism protection device or filter.

In further embodiments, polymer matrices incorporate fibers, such as surface capillary fibers, that can be deployed across the lumen of a patient's vessel to form the polymer matrix. In the fiber-based embodiments described herein, the outer surface of the device may be only generally defined by extrapolating between neighboring fibers along the outer portions of the structure. In general, the fibers can be bound within the structure and may be woven, braided, combined with other polymers or metallic support structures, such as struts, hoops, frames and the like. However, in an embodiment described specifically below, a bundle of fibers fastened at their two ends are deployed in a bent configuration for filtering.

While some specific embodiments are described below with fibers attached at both of their ends, in other embodiments the fibers are attached at one end. In these embodiments, a thicker fiber may be suitable relative to fibers attached at both ends. Fibers attached at one end can be deployed from a sheath or the like such that the fibers spread outward from the axis of a guide structure or catheter following release from the sheath. The sheath can be a catheter-like tube that extends over the fibers to constrain the fibers. In additional or alternative embodiments, the fibers attached at one end can be formed form shape memory polymers that flare outward upon gradual warming in contact with a patient's blood. Embolectomy devices formed with fibers attached at one end can be removed for the patient with the help of a sheath into which the fibers folds for removal form the patient.

For filtering embodiments, the nature of the arrangement of the material across the device generally can be formulated to be consistent with the maintenance of flow through the device while capturing emboli over an appropriate size such that they do not flow past the device. In other embodiments, the fibers are configured to improve the embolectomy function without necessarily providing a high level of emboli capture. The device can comprise a single fiber that folds to form a particular structure, multiple fibers that are arranged various ways, and the structure can comprise one or more fibers combined with one or more additional materials to form the polymer matrix of the device. For example, the fibers can be organized into a bundle that is deployed within the vessel. The device can comprise a plurality of domains with one or both of the domains comprising fibers.

In some embodiments, the polymer matrix comprises surface capillary fibers, although other embodiments can comprise round or other simpler fiber structures. Surface capillary fibers (SCF) fibers are characterized by surface channels or capillaries formed within the surface of the fiber. Surface capillaries are characterized by having a portion of the capillary exposed at the surface of the fiber along the length of the fiber or a portion thereof. Fibers have their usual meaning as structures with a length that is significantly larger than the dimensions along a cross section perpendicular to the length. The capillaries can run along substantially the entire length or a fraction thereof. Due to the presence of the capillaries, a cross section through the fiber at the capillary(ies) has a shape with an edge having changing curvatures.

The surface capillaries result in significant increase in the surface area of the fibers relative to fibers with a smooth surface and the same diameter. In some embodiments, the surface of the fiber has a plurality of surface channels or capillaries along the length of the fiber. An SCF fiber can have surface channels that essentially make up a large fraction of the bulk of the fiber such that little if any of the interior mass of the fiber is not associated with walls of one or more surface capillaries. In particular, the SCF fiber substrate can be formed with a relatively complex cross-sectional geometry. Suitable fibers include, for example, commercially available 4DG™0 fibers (Fiber Innovation Technology, Inc., Johnson City, Tenn.) but would also include new advanced geometries to provide for greater fluid transport or absorption or wetting capabilities.

Suitable approaches for the manufacture of the SCF are described in, for example, U.S. Pat. No. 5,200,248 to Thompson et al. (the '248 patent), entitled “Open Capillary Structures, Improved Process For Making Channel Structures And Extrusion Die For Use Therein,” incorporated herein by reference. The Background section of the '248 patent additionally references a variety of alternative embodiments of approaches for forming fibers with surface channels or capillaries. Any of these approaches can be used to make suitable fibers. Embolism protection devices formed from fibers, such as surface capillary fibers, other approaches for forming fibers with surface channels and methods for characterizing surface capillary fibers are described further in copending U.S. patent application Ser. No. 10/795,131 to Ogle et al., entitled “Fiber Based Embolism Protection Device,” incorporated herein by reference.

As with the fiber length, the thickness of the fibers can be selected appropriately for the particular use of the fiber. Fiber thickness can be measures in several ways. For example, the radius of the fiber can be roughly estimated from the assumption of a circular cross section. Alternatively, one can define an average diameter by taking an average cross section and then averaging the length of segments through the center of the cross section that intersect the circumference of the cross section. Also, calipers can be used to measure thickness, which can be averaged to obtain a value of the diameter. These various approaches at estimating the radius or diameter generally give values of roughly the same magnitude. Also, in the fiber field, a pragmatic way has been developed to characterize fiber thickness without the need to resort to magnification of the fibers. Thus, fiber thickness can be measured in units of denier. Deniers correspond to the number of grams per 9,000 meters of yarn with a larger value corresponding to a thicker fiber. In some embodiments, suitable fibers have diameters from 50 microns to about 5 millimeter, in further embodiments from about 100 microns to about 2 millimeters, and in additional embodiments from about 150 microns to about 1 millimeter. As measured in denier, fibers can have sizes ranging from about 0.1 denier to about 1000 denier in size, in additional embodiments from about 0.5 denier to about 250 denier, in some embodiments from about 1.0 denier to about 200 denier, in other embodiments from about 2.0 denier to about 100 denier and in further embodiments from about 3.0 denier to about 50 denier. A person of ordinary skill in the art will recognize that additional ranges of fiber thickness in diameter measurements or in denier are contemplated and are within the present disclosure. As a specific example, in one specific filter embodiment, the device comprises 480 of 6 denier SCF fibers in a bundle and a crossing profile of 0.033 inches (2.5 French).

Any particular embolectomy device generally can conform to the specific size and shape of the inside of the vessel following a rough size selection for the device. While the particular device size depends on the size of the particular vessel, an embolectomy device following expansion within the vessel of a human patient general can have a diameter perpendicular to the flow direction from about 50 microns to about 35 millimeters (mm), in additional embodiments from about 100 microns to about 9 mm and in further embodiments, from about 500 microns to about 7 mm. A person of ordinary skill in the art will recognize that additional ranges of device diameters within the explicit ranges are contemplated and are within the present disclosure.

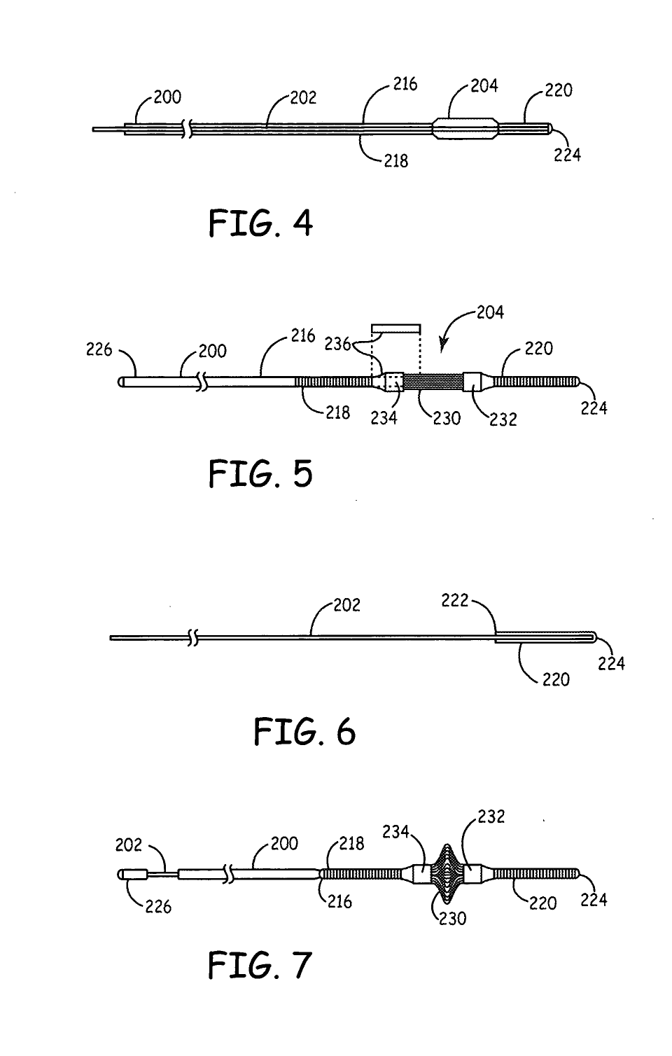

One specific embodiment of a device based on fibers is shown in FIGS. 4-7. In this embodiment, the tether comprises an integrated guiding device. The integrated guiding device comprises a corewire and an overtube. To facilitate guiding of the structure, the integrated guiding device can further comprise a torque coupler that couples angular motion of the corewire and the tube. Torque couplers for an integrated guiding structure are described further in copending U.S. patent application Ser. No. 11/072,001 to Galdonik et al., entitled “STEERABLE DEVICE HAVING A COREWIRE WITHIN A TUBE AND COMBINATION WITH A FUNCTIONAL MEDICAL COMPONENT,” incorporated herein by reference.

In the embodiment of FIGS. 4-7, the integrated guiding device comprises a tube 200, a corewire 202, and an embolism protection structure 204. Referring to the sectional view in FIG. 4 and the side view in FIG. 5, tube 200 has a tapered section 216 at its distal end that mimics the taper on a conventional guidewire. A wire coil 218 is abutted against and secured to tapered section 216. Corewire 202 is covered with a coil 220 at its distal end, as shown in FIG. 6. Coil 220 is connected with solder 222 and a weld 224, although other attachment approaches can be used. Tube 200, corewire 202, wire coil 218, coil 220 and grip 226 can all be formed from stainless steel, although other suitable materials can be used.

In this embodiment, embolism protection device 204 comprises a bundle of SCF fibers 230 attached at first attachment 232 and second attachment 234, as shown in FIGS. 5 and 7. A 0.1 inch long tube 236, which can be formed from polyimide polymer, is located within the second attachment 234 with corewire 202 extending within the tube. The fibers are swaged/crimped/bonded at the two attachments 232, 234 to a diameter of 0.033 inches, such as with radio-opaque bands. After crimping, the fiber bundles can be bonded at each end with an adhesive, such as cyanoacrylate, and/or fused together with heat bonding.

The number of fibers in the bundle generally depends on the desired degree of filtration as well as the thickness of the fibers. In general, the number of fibers can be range from at least 10 fibers, in further embodiments from 25 fibers to 1,000,000 fibers, in other embodiments from 50 fibers to 10,000 fibers and in additional embodiments, from 100 fibers to 5,000 fibers.

The length of the fibers can be selected based on the size of the corresponding vessel. When deployed, the centers of the fibers are projected across the lumen of the vessel. Thus, the unconstrained length of the fibers between attachment structures 232, 234 should be at least double the radius of the vessel. In some embodiments relating to the use of a plurality of fibers to expand within the lumen of a patient's vessel, it is generally appropriate to use fibers that have a length from about 2.2 to about 10 times the vessel radius, in some embodiments from about 2.4 to about 5 times the vessel radius and in further embodiments from about 2.6 to about 4 times the vessel radius. For placement in a human vessel, the fibers generally have a length from about 0.5 mm to about 100 mm, in other embodiments from about 1 mm to about 25 mm, and in further embodiments from about 2 mm to about 15 mm. A person of ordinary skill in the art will recognize that additional ranges of fiber numbers and fiber length within the explicit ranges are contemplated and are within the present disclosure. In general, a particular device is intended for use in a range of vessel sizes.

For fiber based embodiments as well as other embodiments described herein for embolectomy applications, the dimensions of the device should take into account not only the vessel diameter at the location of initial deployment of the device but also the dimensions of the vessel over the distance in which the device is moved during the embolectomy. Thus, the vessel may increase in diameter as the device is moved upstream from the location of initial deployment. In general, the vessel could increase in diameter from zero percent to about 400 percent. Thus, the size of the device should be picked using the guidelines above based on the largest vessel diameter that is encountered. If the tensions within the device tend to expand its diameter, even if they are relatively gentile forces, the device generally gradually adjusts to the increasing vessel size while maintaining superior apposition.

Polymer matrices for use in the medical devices are generally formed from biocompatible polymers. Polymer matrices can be fabricated from synthetic polymers as well as purified biological polymers and combinations thereof. Suitable synthetic polymers include, for example, polyamides (e.g., nylon), polyesters (e.g., polyethylene teraphthalate), polyacetals/polyketals, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Based on desirable properties and experience in the medical device field, suitable synthetic polymers include, in particular, polyether ether ketones, polyacetals, polyamides (e.g., nylons), polyurethanes, polytetrafluoroethylene, polyester teraphthalate, polycarbonates, polysulfone and copolymers and mixtures thereof.

Appropriate polymers also include biological polymers. Biological polymers can be naturally occurring or produced in vitro by fermentation and the like. Suitable biological polymers include, without limitation, collagen, elastin, silk, keratin, gelatin, polyamino acids, cat gut sutures, polysaccharides (e.g., cellulose and starch) and mixtures thereof. Purified biological polymers can be appropriately formed into a polymer material for further processing into fibers.

The polymer matrix can be formed from a radiopaque material to provide for observation of the procedure using x-rays. Suitable radiopaque polymers include, for example, iodinated and brominated polymers, as described for example in U.S. Pat. No. 6,475,477 to Kohn et al., entitled “Radio-Opaque Polymer Biomaterials,” incorporated herein by reference as well as in Kruft et al., “Studies on Radio-Opaque Polymeric Biomaterials With Potential Applications to Endovascular Prostheses,” Biomaterials 17 (18): 1803-1812 (September 1996) and Jayakrishnan et al., “Synthesis and Polymerization of Some Iodine-Containing Monomers for Biomedical Applications,” J. of Applied Polymer Science 44 (4) 743-748 (Feb. 5, 1992). Alternatively, other polymers can be loaded with radiopaque particles to form a radiopaque composite. Radiopacity can be achieved with the addition of markers, such as platinum-iridium or platinum-tungsten or through radio-pacifiers, such as barium sulfate, bismuth trioxide, bismuth subcarbonate, powdered tungsten, powdered tantalum or the like, added to a polymer. Generally, the composite radiopaque material comprises no more than about 60 weight percent radiopaque composition. Radiopaque polymers for the formation of embolic protection devices are described further in copending U.S. Provisional Patent application Ser. No. 60/739,068 to Galdonik et al., entitled “Radiopaque Fibers and Filtration Matrices,” incorporated herein by reference, and the teachings of this provisional patent application can be adapted for the embolectomy devices herein.

Additionally or alternatively, the polymer matrices can be associated with biologically active agents, such as an agent that is effective to dissolve the emboli. Suitable bioactive agents include, for example, thrombolytic (anti-thrombogenic) agents, anti-platelet agents, anti-coagulation agents, growth factors and combinations thereof. Suitable thrombolytic agents include, for example, tissue-type plasminogen activator (tPA), mutated forms of tPA, such as TNK-tPA and YM866, heparin, urokinase, streptokinase, staphylokinase, and the like. Suitable bioactive agents are described further in copending U.S. patent application Ser. No. 10/414,909 to Ogle, entitled “Embolism Protection Devices,” incorporated herein by reference. These bioactive agents can be coated onto the polymer of the embolectomy device alone or with a polymer release agent, such as a bioresorbable polymer. Suitable bioresorbable polymers include, for example, polysaccharides, such as polydextran, cellulose and starch, hydroxyethyl starch, derivatives of gelatin, polyvinylpyrrolidone, polyvinyl alcohol, poly[N-(2-hydroxypropyl)methacrylamide], poly(hydroxyacids), poly(epsilon-caprolactone), polylactic acid, polyglycolic acid, poly(dimethyl glycolic acid), poly(hydroxybutyrate), copolymers thereof and mixtures thereof. Additionally or alternatively, the bioactive agent can be incorporated into the polymer matrix material as it is formed, such that the bioactive agent gradually elutes from the polymer matrix upon deployment.

It has been discovered that particular preparation processes for bundles of fibers can lead to significantly improved uniformity of the performance of the filter device. In particular, the fibers can be twisted within the fiber bundle. In some embodiments, heat is also applied to the fibers. While any degree of twist can be desirable, twist can be applied to the fiber bundle of at least about 5 degrees and in further embodiments from about 180 degrees to about 360 degrees. Furthermore, multiple rotations, for example, about 360 degrees to about 1080 degrees, can further act to increase the density of fibers and may be advantageous. A person of ordinary skill in the art will recognize that additional ranges of twist within the specific ranges above are contemplated and are within the present disclosure. The twist can be applied by fastening one end of the fiber bundle, applying the twist and fastening the other end of the fiber bundle. A suitable torque coupler can facilitate the application of the twist to the fibers since the corewire does not rotate due to tension in the SCF fibers. With the application of a suitable twist, corresponding embolism protection devices have been observed to perform with essentially uniform performance upon deployment in which fibers in the bundle are bent in the deployed configuration. Thus, the twist provides for a commercial device with reproducible performance expected for medical devices in practice.

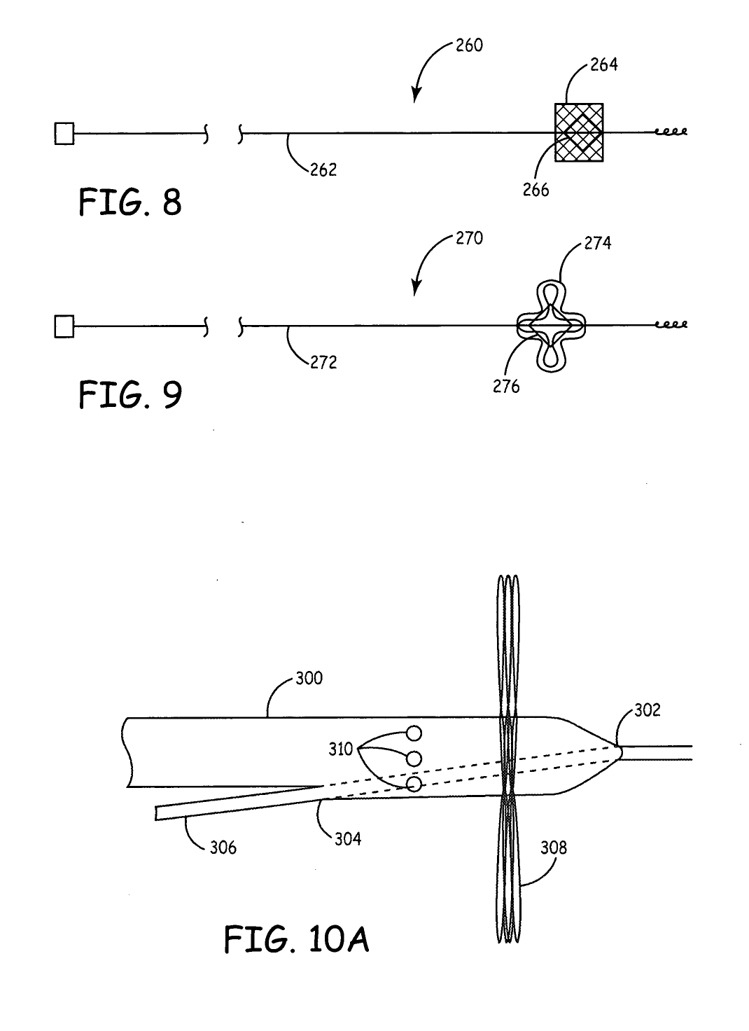

As noted above, a polymer matrix can be associated with a support structure. Such an embodiment is shown in FIG. 8. The embolectomy/filter structure 260 comprises a guide structure 262, a polymer matrix 264 and support elements 266. Support elements 266 can be struts, such a bent metal elements that have a deployed and delivery/recovery configuration. In particular, struts can bend from a straight or partially bent when the polymer matrix is deployed and straighten at least partially when the polymer matrix is recovered. In some embodiments, an actuator is used to transition the polymer matrix and/or the support elements between different configurations. The polymer matrix can be molded around the frame, physically intertwined around the support elements and/or attached using another appropriate technique.

For embodiments with one or more support elements, the polymer matrix can comprise fibers, as shown in FIG. 9. Embolectomy/filter structure 270 comprises a guide structure 272, fibers 274 and support elements 276. Support elements 276 can be struts or other relatively rigid constructs. Fibers 274 can be intertwined around, fastened to or otherwise connected to support elements 276. Support elements 276 can be made from biocompatible metal, a suitable polymer or a combination thereof. Suitable biocompatible metals include, for example, titanium, cobalt, stainless steel, nickel, iron alloys, cobalt alloys, such as Elgiloy®, a cobalt-chromium-nickel alloy, MP35N, a nickel-cobalt-chromium-molybdenum alloy, and Nitinol®, a nickel-titanium alloy.

In alternative system configurations, fiber-based elements can be associated with a catheter structure, such as an aspiration catheter. These fiber-based elements can be located near the distal end of the catheter with suction ports located to collect debris generated from contact between material in the vessel and the fiber elements. For example, a series of suction ports can be located around the circumference of the catheter a short distance proximal to the fibers. Additionally or alternatively, the catheter can have a suction port at the distal end of the catheter.

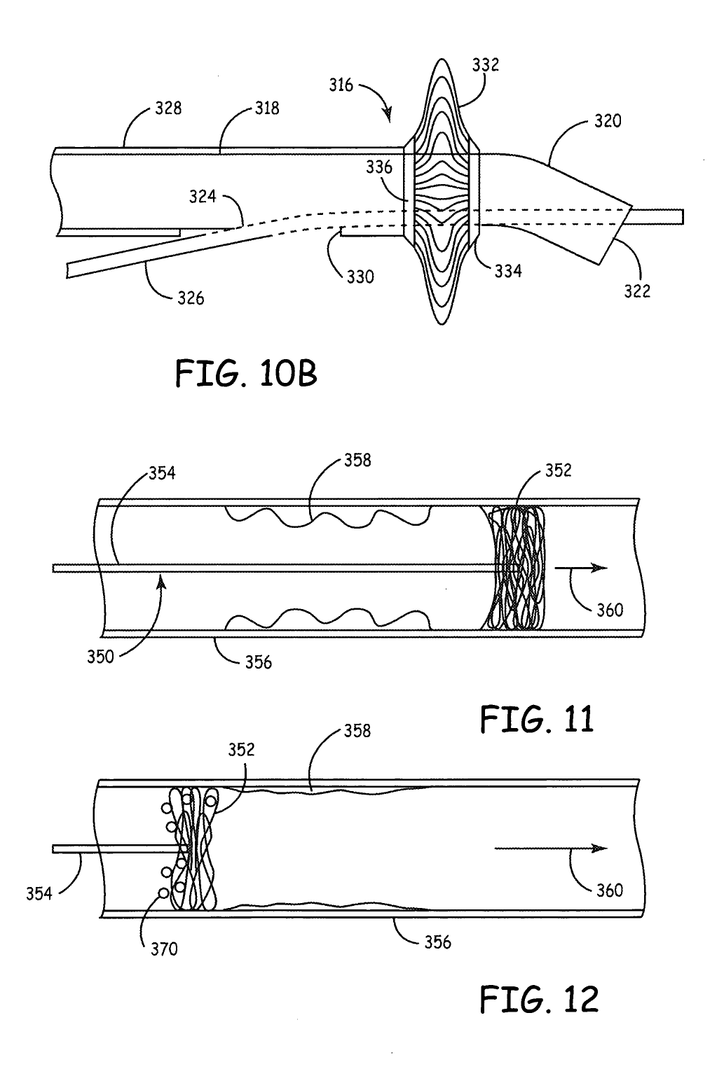

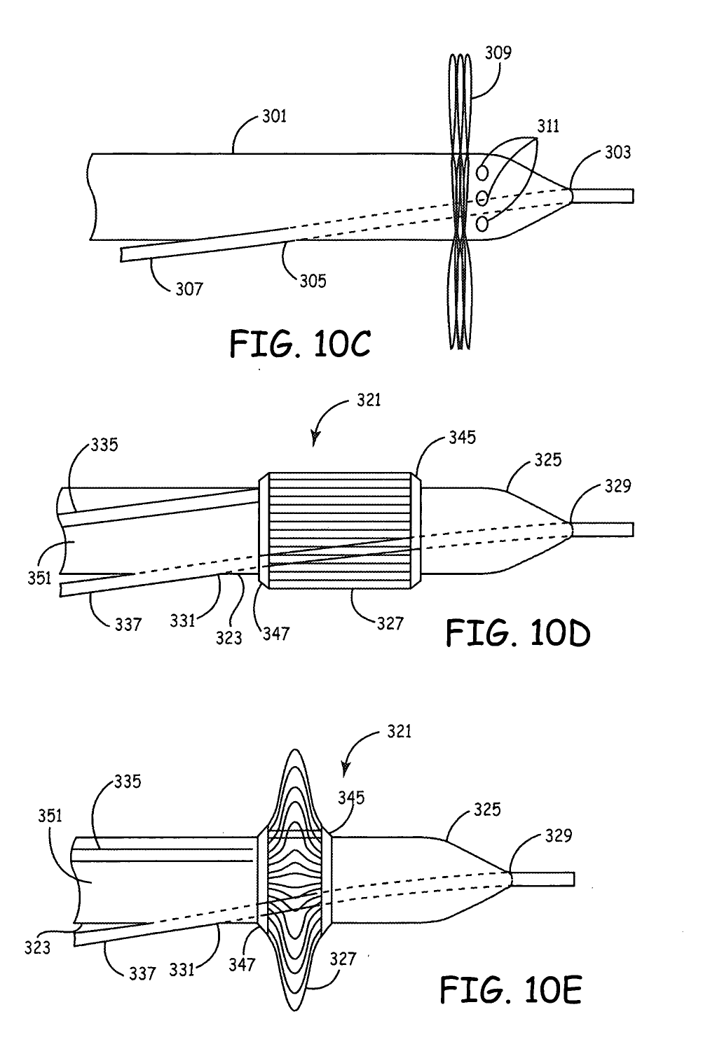

A first representative embodiment is shown in FIG. 10A. In this embodiment, embolectomy catheter 300 has a distal guide port 302 and a rapid exchange guide port 304. A guide structure 306 can traverse between guide ports 302, 304. In alternative embodiments, an over-the-wire configuration can be used. Fibers 308 extend outwardly around the circumference of the catheter. Suction ports 310 are located around the circumference of catheter 300 in a proximal direction relative to fibers 308. In this embodiment, there are six aspiration ports with three shown and three on the opposite side of the catheter not shown. A different number, size and position of suction ports can be used. For example, suction ports may be staggered along the length of the catheter rather than being along the same circumference. In alternative embodiment, the fibers and/or the suction ports may not be circumferentially positioned. In other words, the catheter does not need to be symmetric since the fibers can be positioned only on one side of the catheter. Similarly, the catheter tip may be curved with the fibers positioned, for example, to orient in the direction of the curve.

Catheter 300 and similar devices are suitable for percutaneous and/or surgical procedures. For the delivery of catheter 300, the fibers can be restrained in a sheath or the like. The sheath can be removed to free the fibers. Non-fiber based embolectomy devices can be similarly constrained for delivery with a sheath. The fibers can be formed with tension or shape memory such that the freed fibers take a desirable configuration when freed to perform the embolectomy procedure.

In other embodiments, the catheter can be formed with moveable elements such that deployment of the fibers can be based on the relative movement of the actuation elements in analogy with device shown in FIGS. 4-7. Then, the fibers can be deployed and collapsed as appropriate. Such an alternative embodiment is shown in FIG. 10B.

Referring to FIG. 10B, catheter structure 316 comprises a tubular element 318 with a bent tip 320 and a distal opening 322. A rapid exchange port 324 provides for insertion of guide structure 326 into tubular element 318, and guide structure 326 can exit tubular element 318 at distal opening 322. Overtube 328 rides over tubular element 318. Overtube 328 can have a slit or similar structure 330 to provide for the relative movement of overtube 328 and tubular element 318 without interference from guide structure 326. Since overtube 328 generally does not need to hold liquid since tubular element 318 can define a suction lumen or other central lumen, overtube 328 can have a structure that is only tubular in the sense of being able to ride over tubular element 318 and transmit longitudinal movement from the proximal end of the device to the distal end of overtube 328. Fibers 332 attach at a first end to tubular element 318 at first band 334, and fibers 332 attach at a second end to overtube 328 at second band 336. Relative movement of overtube 328 and tubular element 318 can be used to transition fibers 332 between a deployed, flared out configuration and a straighter configuration for delivery into and removal from a vessel.

Another alternative embodiment is shown in FIG. 10C, which is particularly appropriate for a blockage in a vessel that is difficult to pass. In this embodiment, catheter structure 301 comprises a distal guide port 303 and a rapid exchange port 305. Guide structure 307 extends between distal guide port 303 and rapid exchange port 305. Fibers 309 extend outwardly around the circumference of the catheter. In this embodiment, suction ports 311 are located around the circumference of the catheter near the base of fibers 309 on the distal side of catheter relative to the fibers. This embodiment is suitable for treating blockages that are difficult to pass since the catheter has a relatively pointed tip and aspiration ports near the distal end so that emboli generated at the distal edge of the fibers can be removed through the suction ports. Other configurations of suction ports on the distal side of the embolectomy element can be used as desired to replace the particular embodiment shown in FIG. 10C.

An embodiment in which suction can be applied through a gap in the catheter structure is shown in FIGS. 10D and 10E, in which an embolectomy device connects the two portions of the catheter. Referring to FIG. 10D, embolectomy catheter 321 comprises a tubular section 323, tip section 325 and emebolectomy device 327 connecting tubular section 323 and tip section 325. Tip section 325 comprises a distal guide port 329. Tubular section 323 has a rapid exchange guide port 331. Pullwire 335 extends within the lumen of tubular section 323. Guide structure 337 extends between distal guide port 329 and rapid exchange guide port 331 and passes within embolectomy device 327.

Embolectomy device 327 comprises a deformable polymer matrix 343, distal attachment element 345 and proximal attachment element 347. Deformable polymer matrix 343 can comprise a plurality of fibers attached at their respective ends with distal attachment element 345 and proximal attachment element 347. Proximal attachment element 347 connects polymer matrix 343 with tubular section 323. Distal attachment element 345 connects polymer matrix 343 with tip section 325. Distal attachment element 345 and proximal attachment element 347 can comprise a band of radiopaque material, adhesive and/or additional fastening structures, as appropriate. Pull wire 335 connects with distal attachment element 345. Embolectomy catheter 321 can comprise a plurality of pull wires or the like and/or other structures that provide for actuating embolectomy device 327.

Referring to FIG. 10E, embolectomy device 321 is shown in a deployed configuration which is obtained by pulling pull wire 335 in a proximal direction relative to tubular section 323. In the deployed configuration, polymer matrix 343 flares outward relative to the axis of catheter 321 so that the polymer matrix 343 is configured for performing the embolectomy procedure. Due to the gap between tubular element 323 and tip element 325 along with the porosity of polymer matrix 343, an effective suction port is formed at the end of tubular element 323 such that suction can be applied through a suction lumen 351 within tubular element 323. Thus, suction can be used to remove any debris that passes through polymer matrix 343. Pushing of pull wire 335 can transition polymer matrix 343 to a recovery configuration similar to the deployment configuration shown in FIG. 10D.

Procedures for Using Embolectomy Devices

The general procedure for performing the embolectomy comprises the placement of the embolectomy device within the patient's vessel at a selected location. Once the embolectomy device is at the selected location, the embolectomy device is moved to dislodge emboli for removal. During the embolectomy procedure, flow through the vessel generally can be maintained with little or no pressure drop across the embolectomy device. In some embodiments, the embolectomy device has a polymer matrix that conforms to the vessel wall such that variation and/or irregularities in the vessel wall along the length of the vessel can be accommodated on a continuous basis by the embolectomy device as it is moved due to continuous adjustment to the vessel wall. For example, some fiber-based embolectomy devices described herein have bent fibers from a bundle contacting the walls of the vessel in which the flexible fibers conform under contact to the shape of the vessel wall. While dislodged emboli may collect on and/or within the polymer matrix, suction can be applied during the embolectomy procedure and/or during the recovery of the embolectomy device from the vessel. Additionally or alternatively, a separate filter/embolic protection device can be used to collect emboli generated in the procedure.

In general, the devices and procedures described herein can be used within any reasonable vessel within a patient. The embolectomy procedures can be performed in a less invasive, percutaneous format or a surgical format. In a less invasive format, guide catheters, hemostatic valves and other devices to facilitate catheter use can be adapted for use with the embolectomy systems described herein. For example, generally heart procedures involve an incision in the inner thigh to access an artery that leads to the heart. Suitable visualization approaches can be used to position the tip of the catheter. The thrombus can be located prior to and/or during the embolectomy procedure.

In a surgical format, the vessel is exposed during the procedure and the device is inserted through an incision in the exposed vessel. Thus, the embolectomy procedures are well suited for use during carotid endartorectomy to remove emboli from vessels flowing to the patient's brain. The surgical systems are also suitable, for example, for use in procedures involving the placement of vascular grafts, such as coronary artery bypass grafting, in which the graft is subject to anastamose through an incision as part of the procedure. However, more generally, the surgical systems can be used for any procedures performed on an exposed vessel. As in the percutaneous, less invasive procedures, the embolectomy device is removed from the vessel at an appropriate point in the procedure.

In some specific embodiments, after the vessel is exposed, a purse-string suture is tied around the proposed access site. A purse-string suture is a continuous suture line that can be tightened at a selected time to cinch the hole closed. Then, a cannula is inserted into the sidewall of the vessel, either with or without suction. A wire can be advanced into the vessel to confirm that the cannula is completely into the vessel. Once this is verified, the wire is removed. This procedure with the cannula is referred to commonly as the Seldinger Technique. After the embolectomy device is removed, the purse-string suture is closed and tied.

The movement of the embolectomy device can dislodge thrombus/emboli for removal from the vessel. In particular, if the embolectomy device is moved in the upstream direction the movement can encourage collection of the emboli in the polymer matrix if the polymer matrix is suitable for filtration. In general, the embolectomy device can be moved in either an upstream or downstream direction, and as part of an overall procedure, the device can be moved sequentially in both an upstream and a downstream direction, in either order and if desired, reversing directions a plurality of times to achieve desired levels of emboli removal. In some embodiments, the embolectomy device is moved at least about 2 millimeters and in further embodiments, from about 2.5 millimeter to about 5 centimeters. A person of ordinary skill in the art will recognize that additional ranges of motion within these explicit ranges are contemplated and are within the present disclosure.

Suction can be used to remove some or all of the emboli generated in the embolectomy procedure. Suction can be applied at one or more portions of the procedure. In particular, suction can be applied while an embolectomy device is being moved in the vessel to capture at least some of the emboli shortly after they are generated. To the extent that at least some of the emboli are trapped during the procedure by the embolectomy device and/or a separate filter device, suction can also be user during the removal from the vessel of the embolectomy device and/or a separate filter device.

In general, it can be desirable to apply suction during the recovery of the embolectomy device from the vessel to collect any emboli that may be dislodged from the embolectomy device during the recovery process. Similarly, suction can be applied during recovery of a separate filter to capture any emboli that are released from the filter during the removal process. The embolectomy device and/or a separate filter device can be transitioned from a deployed configuration to a lower profile recovery configuration prior to removal. Suction can be similarly applied during the conversion of a device to the recovery configuration. In the lower profile recovery configuration, the embolectomy device and/or the separate filter device can be withdrawn partially or completely within the distal end of an aspiration catheter for removal from the patient. Additionally or alternatively, suction can be applied as the device is drawn into the catheter. Use of aspiration during the recovery of a filter/embolic protection device is described further in copending U.S. patent application Ser. No. 10/854,920 to Galdonik et al., entitled “Emboli Filter Export System,” incorporated herein by reference.

As noted above, in addition or as an alternative to applying suction during recovery of the device, suction can be applied while the device is being moved within the vessel. This suction procedure during movement of the filter can be advantageous for all passive embolectomy devices used in performing an embolectomy and not just for particular embodiments of passive embolectomy devices described herein. Passive embolectomy devices refer to devices that are structures that are physically moved through the vessel to dislodge emboli through mechanical contact or interference, in contrast with devices that deliver energy, such as rotational cutting energy, electrical energy, laser energy, or vibrational/acoustic/ultrasound energy, at emboli in a powered embolectomy device.

In general, the aspiration catheter can be held in a fixed position while the embolectomy device is moved through the vessel, or the aspiration catheter can be moved along with the embolectomy device when the embolectomy device is moved. Suitable combinations can also be performed such that the aspiration catheter is moved for a portion of the time that the embolectomy device is moved within the vessel. Similarly, there can be sequential movement of the aspiration catheter and the embolectomy device. For example, the embolectomy device can be moved a selected distance and stopped, then the aspiration catheter is moved a selected distance and stopped, and then the embolectomy element is moved again. These various optional steps can be combined and mixed as selected.

In general, aspiration can be applied the entire time that the embolectomy device is moved or for a fraction thereof. The rate of moving the embolectomy device can balance several factor include, for example, the effectiveness of dislodging emboli, the aspiration rate and the blood vessel flow rate through the vessel. Similarly, the total distance traveled by the embolectomy device within the vessel can balance the disruption of the natural flow through the vessel as well as the size of the region of the vessel to be treated. The procedure can be divided into time segments with movement of the embolectomy device with aspiration for a first period of time, and then stopping the movement and aspiration for a rest period to allow for the return of unrestricted flow through the vessel before movement and aspiration are resumed. These procedures can be tailored to the particular situation for the patient.

As noted above, an embolectomy device can be used along with a separate filter to assist with the capture of emboli for removal from the patient. Depending on the construction of the devices, the embolectomy device and the filter device may be deployed simultaneously or separately. To facilitate the capture of emboli, the filter is generally deployed downstream from the embolectomy device such that the flow moves emboli to the filter. In general, the filter is deployed prior to the movement of the embolectomy device in the vessel to disrupt emboli. Also depending on the construction of the devices, the filter may or may not remain fixed while the embolectomy device is moved. The filter can remain deployed at least until movement of the embolectomy procedure is completed.

For removal from the patient, the embolectomy device and/or the filter can be converted to a recovery configuration. For appropriate embodiments, the embolectomy device and the filter device can be transitioned to a recovery configuration simultaneously or sequentially. Suction can be applied, if desired, during the conversion of the embolectomy device and/or a filter to the recovery configuration to remove any emboli that become dislodged from the devices as they transition to the recovery configuration. Also, it can be desirable to withdraw the embolectomy device and/or the filter at least partially into a catheter for removal from the patient.

In general, associated with any of the procedures described herein, liquids can be delivered into the vessel to facilitate the procedure. For example, physiologically buffered saline or the like can be delivered into the vessel to help flush emboli within the vessel. Sterile buffered saline is well known in the art. Similarly, bioactive agents, such as heparin or other anti-thrombogenic agents, can be delivered into the vessel to help dissolve or loosen emboli. A bioactive agent can be delivered with a suitable carrier such as buffered saline or other carriers known in the art. The buffered saline and/or bioactive agents can be delivered through a catheter or the like into the vessel. Suitable volume of liquids and doses of bioactive agents may depend on the size of the vessel and can be determined by a physician based on knowledge in the art.

An embodiment of the general procedure is depicted schematically in FIGS. 11 and 12. Referring to FIG. 11, embolectomy device 350 comprises a polymer matrix 352 and a tether 354. Polymer matrix 352 is positioned within a patient's vessel 356 at a position downstream from thrombus/emboli 358. Flow arrow 360 depicts the direction of natural flow through the vessel. As shown in FIG. 12, polymer matrix 352 is translated in an upstream direction relative to its position in FIG. 11. As a result of the translation of polymer matrix 352, thrombus/emboli 358 is reduced or eliminated in association with the walls of vessel 356 over the portions of the vessel swept with polymer matrix 352. Correspondingly, in this embodiment emboli 370 become entrapped within polymer matrix 358. Embolectomy device 350 and entrapped emboli can be removed appropriately from vessel 356.

In some embodiments, the embolectomy device is delivered into the patient's vessel in a low profile configuration. A low profile configuration of the embolectomy device is less likely to disrupt flow during delivery, is less likely to become entangled within the vessel during delivery and is easier to maneuver to the desired location. Additionally or alternatively, it may be desirable to take specific steps to decrease or eliminate the chance of release of emboli from the embolectomy device upon recovery of the embolectomy device from the vessel. In particular, in some embodiments, the embolectomy device is transformed from a deployed configuration to a recovery configuration. The embolectomy device in a recovery configuration can be drawn into a distal compartment of a catheter to remove the embolectomy device from the flow during removal from the vessel. Furthermore, in some embodiments, the recovery catheter can supply suction to capture any released emboli such that they are removed from the flow rather than releasing the emboli downstream. Capture catheters with suction are described further in copending U.S. patent application Ser. No. 10/854,920 to Galdonik et al., entitled “Emboli Filter Export System,” incorporated herein by reference.

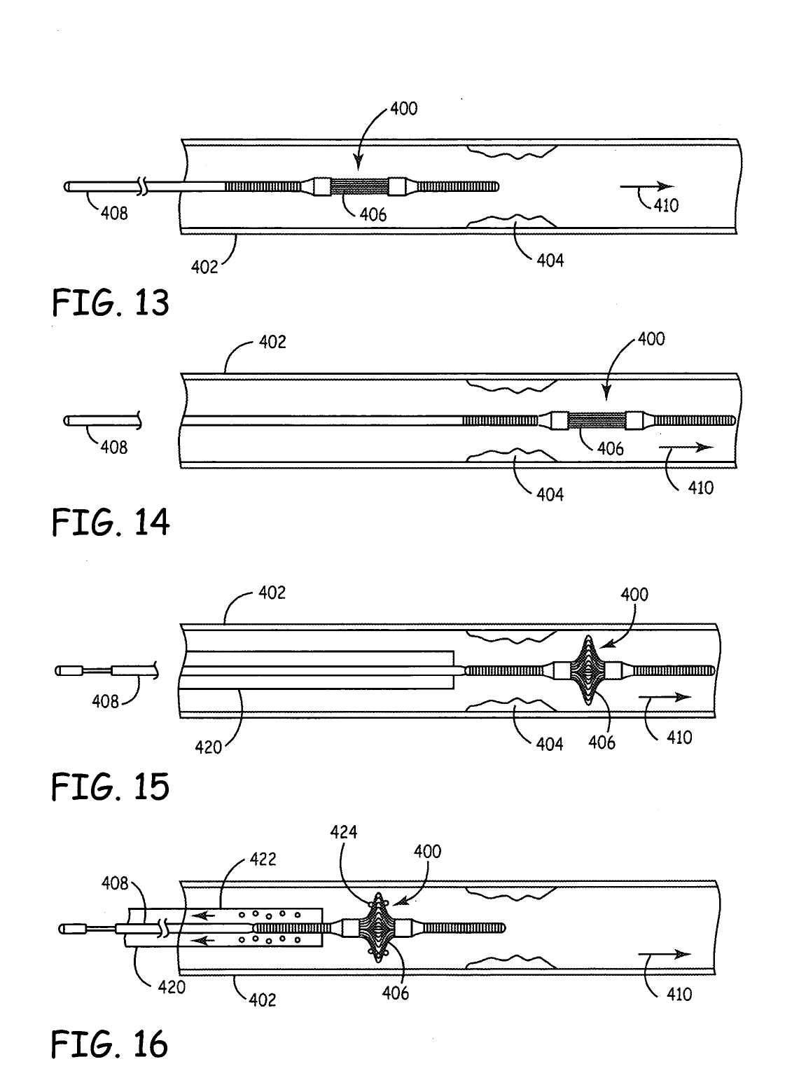

An embodiment of an embolectomy procedure with suction is depicted in FIGS. 13-19 based on the embolectomy device shown in FIGS. 4-7. Referring to FIG. 13, emolectomy device 400 is delivered within patient's vessel 402 past thrombus/emboli 404. As shown in FIG. 14, polymer matrix 406 in a low profile delivery configuration is positioned downstream from thrombus/emboli 404. Once in position, polymer matrix 406 can be deployed using integrated guide structure 408 (see FIG. 7) into a deployed configuration, as shown in FIG. 15. The natural flow direction in the vessel is indicated with flow arrow 410. As shown schematically in FIG. 15, the fibers in a bent configuration contact the walls of the vessel. In actual use, the fibers are generally long enough so that they are deflected through their contact with the vessel wall such that a conforming polymer matrix is located adjacent the wall of the vessel. An aspiration catheter 420 can be positioned within the vessel to collect emboli dislodged during the procedure.

Referring to FIG. 16, the deployed polymer matrix is moved within vessel 402 in a downstream direction to dislodge and capture thrombus/emboli 422. The embolectomy device is stopped at a selected downstream position with reduced or eliminated thrombus 404 along the walls of the vessel. Suction is applied through aspiration catheter 420 during at least a portion of the time that the filter is moved. Loose emboli 422 are collected within the aspiration catheter while other emboli 424 remain associated with polymer matrix 406.

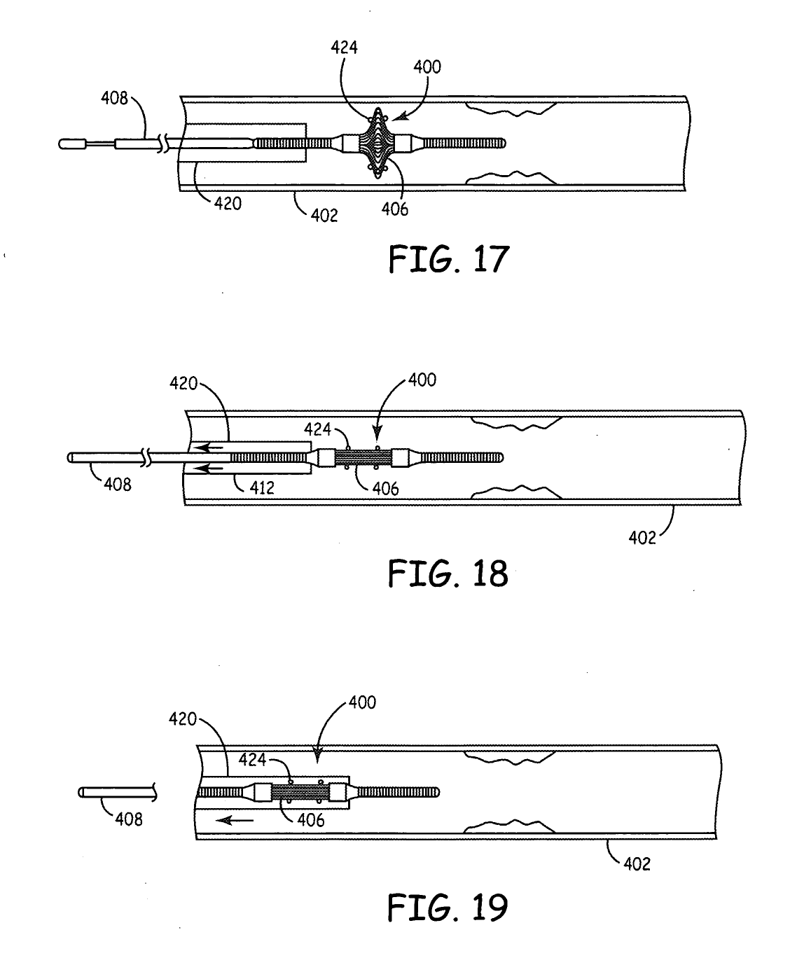

FIGS. 17-19 depict the steps for the removal of the embolectomy device 400 from vessel 402. Referring to FIG. 17, an aspiration catheter 420 is positioned over integrated guide device 408 near polymer matrix 406. Polymer matrix 406 can be converted to a recovery configuration while suction is applied through aspiration catheter, as shown in FIG. 18. Emboli 422 released from polymer matrix 406 when it is converted to the recovery configuration can be captured by aspiration catheter 420, while other emboli 424 remain within polymer matrix 406. Suction within the aspiration catheter is depicted with flow arrows in FIG. 18. Suction can be continued as the polymer matrix 406 is drawn into the end of aspiration catheter 420, as shown in FIG. 15. With the polymer matrix 406 within aspiration catheter 420, aspiration catheter 420 can be removed from the patient as indicated by the arrow in FIG. 19.

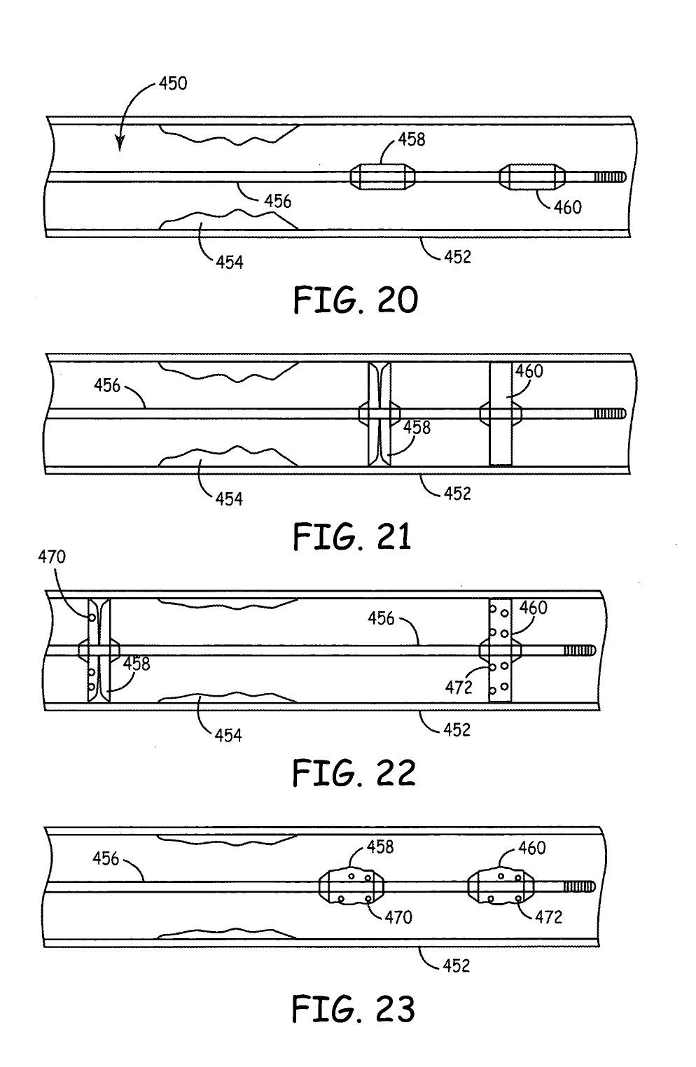

As noted above, the embolectomy device can be used with a separate filter. Use of one embodiment of a system with both an embolectomy device and a filter device is depicted in FIGS. 20-23. In this embodiment, the embolectomy device and the filter device can move relative to each other. Use of other devices in which the embolectomy device is fixed relative to the filter device follows similarly. Referring to FIG. 20, embolectomy system 450 is deployed within vessel 452 having thrombus/emboli 454 at a lesion of the like. Embolectomy system 450 comprises a guide structure 456, embolectomy device 458 and filter 460. As shown in FIG. 20, embolectomy device 458 and filter 460 are delivered in a low profile configuration. Generally, filter 460 is positioned downstream from thrombus/emboli 454, although embolectomy device 458 can be positioned initially either upstream or downstream from thrombus/emboli 454. Referring to FIG. 21, embolectomy device 458 and filter 460 can be converted to a deployed configuration sequentially or simultaneously.

As depicted in FIG. 22, embolectomy device 458 can be moved past thrombus/emboli 454 to dislodge emboli for collection and subsequent removal. Some emboli 470 can become associated with embolectomy device 458 and/or other emboli 472 are collected with filter 460. Embolectomy device 458 can be moved past thrombus/emboli 454 once or a plurality of time with the motion being in an upstream and/or downstream direction. For removal from vessel 452, embolectomy device 458 and/or filter 460 can be transitioned to a lower profile recovery configuration, as shown in FIG. 23. The recovery configuration can approximate the delivery configuration. Emboli 470, 472 may remain associated, respectively, with embolectomy device 458 and filter 460 when the devices are transitioned to their recovery configurations. This transition to the recovery positions can be performed sequentially or simultaneously.

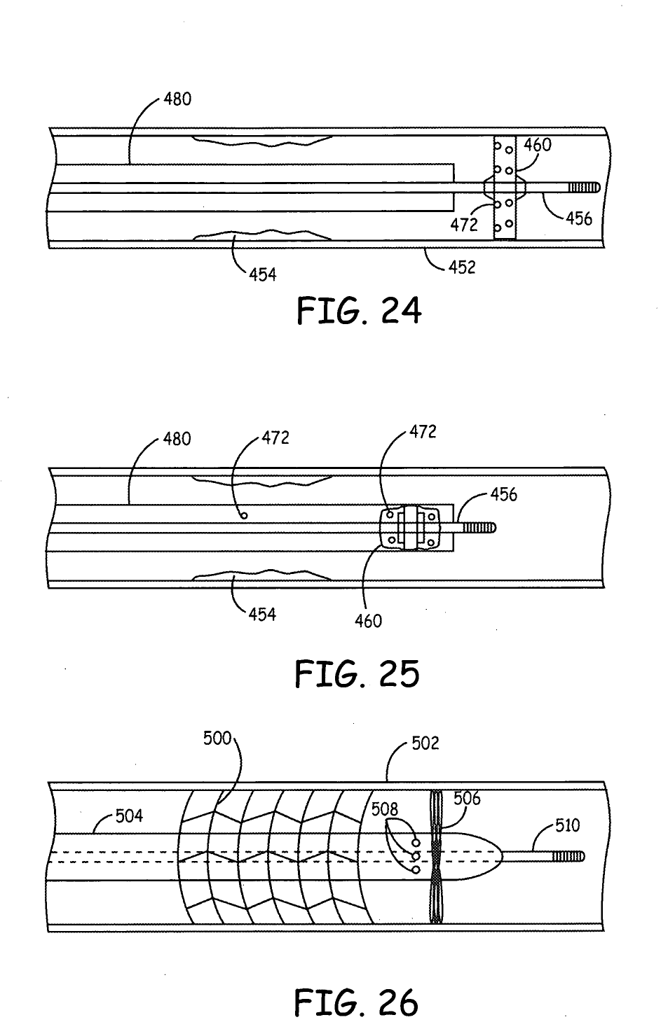

An alternative to the recovery embodiment depicted in FIG. 23 for filter element 460 is shown in FIGS. 24 and 25. Referring to FIG. 24, embolectomy device 458 has been removed by an appropriate process, which may involve a catheter, such as an aspiration catheter. Sheath 480 is positioned in vessel 452 for the removal of filter 460. As shown in FIG. 25, filter 460 is drawn into sheath 480 for removal from the patient. In some embodiments, contact with sheath 480 collapses the filter, although in some embodiments, the filter has an actuation component to facilitate its transition to a lower profile format. In some embodiments, sheath 480 is an aspiration catheter such that any emboli 472 that release from the filter during recovery are drawn into the sheath, as shown in FIG. 25.

Vascular stents have gained wide use. Following delivery of a stent it can be desirable to remove debris within the stent that is loosened from stent delivery but remains associated with the stent. Suitable embolectomy procedures can be performed with the various embolectomy devices described herein. A representative procedure is shown in FIG. 26 based on the device similar to that of FIG. 10A, although in an over the wire format. Stent 500 has been placed within vessel 502. Embolectomy catheter 504 has been positioned within vessel 502 with a deployed embolectomy element 506. As shown in FIG. 26, embolectomy element 506 is placed downstream from stent 500. Aspiration can be performed through aspiration ports 508. In this embodiment, embolectomy catheter 504 is pulled in an upstream direction along guide structure 510 to dislodge and remove through aspiration any debris along the interior of stent 500.

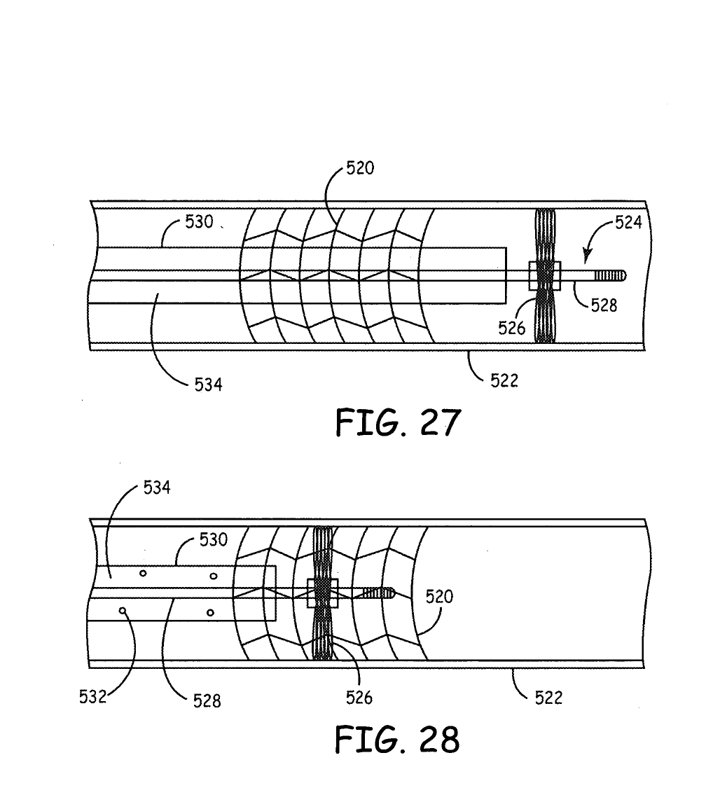

Another embodiment for removing debris from the interior of a stent is shown in FIGS. 27 and 28. In this embodiment, an embolectomy device is used together with an aspiration catheter, which is moved together with the embolectomy device through the interior of the stent. Referring to FIG. 27, stent 520 is shown within vessel 522. Embolectomy structure 524 comprises an embolectomy device 526 associated with a guide structure 528. Embolecotmy device 524 can be the device of FIGS. 4-7 or other suitable device or devices described herein. Aspiration catheter 530 is shown positioned near embolectomy device 524, which is shown in a deployed configuration. As shown in FIG. 27, embolectomy device 526 is positioned downstream from stent 520. Referring to FIG. 28, embolectomy device 526 together with aspiration catheter has been pulled within stent 530 in a proximal direction. Emboli 532 loosened from the inside of the stent due to contact with embolectomy device 526 can be aspirated from vessel 522 through the aspiration lumen 534. Generally, the process of FIGS. 27 and 28 would be continued with embolectomy device 526 and aspiration catheter 530 pulled through stent 530 at its proximal side. Additionally or alternatively, deployed embolectomy device 526 and aspiration catheter 530 can be pushed through the interior of stent 520 in a distal direction.

As will be recognized by a person of ordinary skill in the art, many variations on this procedure can be used to perform an embolectomy within the inside of the deployed stent using an appropriate embolectomy device to achieve the loosening and removal of associated thrombus, based on the teachings herein.

While in principle emboli can remain associated with the embolectomy devices for removal from the patient along with the embolectomy devices, suction can be used to capture emboli that may be released during the removal process. Thus, in some embodiments, suction is applied while the devices are transitioned to their recovery configurations and/or when the devices are pulled into a catheter for removal. In particular embodiment, the embolectomy device is first transitioned to its recovery configuration and pulled into the distal end of a catheter while the filter is still deployed. Suction may or may not be applied at one or more steps of the process of recovering the embolectomy device. Then, the filter is similarly recovered, and suction can be applied while the filter is converted to a recovery configuration and/or while the filter is at least partially withdrawn into a catheter.

The embolectomy systems described herein provide for improved capability to perform an embolectomy procedure to remove significant quantities of thrombus/emboli form a patient's vessel with little or no disruption of flow through the vessel during the procedure. The use of a relatively soft polymer matrix structure avoids vessel damage while introducing the ability to conform to any reasonable vessel geometries to provide superior apposition. The devices described herein can be deployed with a small profile and with superior tracking due to its flexibility. When recovered with an aspiration catheter, the devices can be correspondingly removed from the patient with reduced or eliminated risk of releasing emboli during the recovery. Furthermore, use of suction provides that the devices are not overloaded and that debris generally does not spill into side branches of the vessel. These improved procedures can be combined with other procedures, such as a thrombectomy procedure, an angioplasty procedure and/or a stent delivery procedure.

Distribution and Packaging

The medical devices described herein are generally packaged in sterile containers for distribution to medical professionals for use. The articles can be sterilized using various approaches, such as electron beam irradiation, gamma irradiation, ultraviolet irradiation, chemical sterilization, and/or the use of sterile manufacturing and packaging procedures. The articles can be labeled, for example with an appropriate date through which the article is expected to remain in fully functional condition. The components can be packaged individually or together.

Various devices described herein can be packaged together in a kit for convenience. The kit can further include, for example, labeling with instruction for use and/or warnings, such as information specified for inclusion by the Food and Drug administration. Such labeling can be on the outside of the package and/or on separate paper within the package.

The embodiments above are intended to be illustrative and not limiting. Additional embodiments are within the inventive concepts. Although the present invention has been described with reference to particular embodiments, workers skilled in the art will recognize that changes may be made in form and detail without departing from the spirit and scope of the invention. The incorporations by reference above of the indicated references are limited to the extent to exclude subject matter that is directly contradictory to the explicit disclosure herein.

What is claimed is:

1. A method for the removal of thrombus with an embolectomy device comprising a guide structure and a plurality of fibers operably connected to the guide structure, the method comprising moving the embolectomy device within a vessel of a patient to dislodge thrombus with the embolectomy device wherein suction is applied within the vessel to remove at least a portion of the dislodged thrombus.

2. The method of claim 1 wherein the suction is applied for at least a portion of the time while the filter element is moving.

3. The method of claim 1 wherein the suction is applied for the entire time that the filter element is moved.

4. The method of claim 1 wherein the fibers form a three dimensional filtration matrix.

5. The method of claim 1 further comprising applying suction while the embolectomy device is transformed into a recovery configuration and drawing the filter element in its recovery configuration into a distal opening of an aspiration catheter.

6. The method of claim 5 wherein suction is applied for at least a portion of the time when the embolectomy device is brought into the catheter.

7. The method of claim 1 wherein the embolectomy device is moved in a continuous fashion across a region where thrombus is thought to be present.

8. The method of claim 1 further comprising deploying the embolectomy device at a location downstream from a location identified with thrombus.

9. The method of claim 1 wherein the guide structure comprises an integrated guiding structure comprising a corewire within an overtube and further comprising actuating the fibers into a deployed configuration prior to the movement of the embolectomy device to associate thrombus wherein the actuation of the fibers comprises moving of the corewire relative to the overtube.

10. The method of claim 9 further comprising after movement of the embolectomy device to associate thrombus, transitioning the fibers to a recovery configuration through the movement of the corewire relative to the overtube in an opposite direction as used to actuate the fibers.

11. The method of claim 1 wherein the plurality of fibers comprises a bundle of fibers.

12. The method of claim 1 wherein the fibers are associated with a support structure.

13. The method of claim 1 wherein the embolectomy device further comprises a porous filtration sleeve operably connected to the guide structure distal to the fibers.

14. The method of claim 1 further comprising delivering a liquid into the vessel.

15. The method of claim 1 wherein the emboletomy device comprises a bioactive agent.

16. A method for the removal of thrombus with an embolectomy device comprising a guide structure, an embolectomy device comprising a polymer matrix and a filter comprising a three-dimensional filtration matrix having a deployed configuration with the filtration matrix extending across the lumen of a vessel, the method comprising moving the embolectomy device within a vessel of a patient to dislodge thrombus while the filter is in its deployed configuration at a fixed location within the vessel.

17. A biocompatible filtration device comprising a guide structure having a proximal end and a distal end, a three dimensional filtration matrix connected near the distal end of the guide structure and a porous filtration membrane connected to the guide structure distal to the three dimensional filtration matrix.

18. The biocompatible filtration device of claim 17 wherein the guide structure comprises an integrated guide structure having an overtube and corewire extending through the overtube.

19. The biocompatible filtration device of claim 18 wherein the three dimensional filtration matrix changes configuration in response to relative movement of the overtube and the corewire.

20. The biocompatible filtration device of claim 17 wherein the three dimensional filtration matrix comprises a plurality of fibers.

21. The biocompatible filtration device of claim 17 wherein the three dimensional filtration matrix comprises woven fibers.

22. The biocompatible filtration device of claim 17 wherein the three dimensional filtration matrix comprises a hydrogel.

23. The biocompatible filtration device of claim 17 wherein the porous filtration sleeve comprises a polymer or metal sheet with holes through the sheet.

24. The biocompatible filtration device of claim 17 wherein the porous filtration sleeve comprises struts attached proximally to a porous sheet, wherein the struts mediate deployment of the sleeve.

25. The biocompatible filtration device of claim 17 wherein the three dimensional filtration matrix is in contact with a filtration surface of the porous filtration sleeve.

26. A biocompatible filtration device comprising a guide structure having a proximal end and a distal end, a three dimensional filtration matrix connected near the distal end of the guide structure and a support structure interfaced with the three dimensional filtration matrix to form a filter element having a deployed configuration and a recovery configuration, wherein in the deployed configuration the three dimensional filtration matrix extends outward from the support structure with respect to the filtering function of the device relative to the axis of the guide structure.

27. A biocompatible embolectomy device comprising a guide structure having a proximal end and a distal end, a plurality of polymeric fibers connected near the distal end of the guide structure and a support structure wherein the fibers interface with the support structure to reinforce the fibers, wherein the fibers have a deployed configuration and a recovery configuration and wherein in the deployed configuration the polymeric fibers extend outward from the support structure relative to the axis of the guide structure.

28. An embolectomy device comprising a tubular catheter having a distal end and a proximal end with a lumen extending from the proximal end to an aspiration port at or near the distal end, an aspiration apparatus operably connected at or near the proximal end of the tubular catheter and a plurality of fibers connected to the surface of the catheter at a localized section within 10 centimeters from the distal end of the catheter.

29. The embolectomy device of claim 28 wherein the fibers extend outward when unconstrained.

30. The embolectomy device of claim 28 wherein the fibers connect a proximal segment and a distal segment of the catheter and wherein a pullwire connect the proximal end with the distal segment.

31. A method for removing debris from a vessel of a patient, the method comprising sweeping an embolectomy device through a deployed stent within a patient's vessel and applying suction to remove debris loosened by the embolectomy device.
